(19) United States
(12) Patent Application Publication
(10) Pub. No.: US 2012/0309733 A1
(43) Pub. Date: Dec. 6, 2012

(54) Composition

(75) Inventors: Wanlin Chang, Bangkok (TH); Amit Jayaswal, Shanghai (CN); Zheng Rong Li, Shanghai (CN); Busaraporn Samran, Bangkok (TH)

(73) Assignee: CONOPCO, INC., D/B/A UNILEVER, Englewood Cliffs, NJ (US)

(21) Appl. No.: 13/571,422

(22) Filed: Aug. 10, 2012

Related U.S. Application Data
(62) Division of application No. 13/266,241, filed on Dec. 8, 2011.

(30) Foreign Application Priority Data
May 8, 2009 (EP) EP09159807

(51) Int. Cl. A61K 31/555; A61P 31/10; A61P 17/00

(52) U.S. Cl. 514/188

(57) ABSTRACT

Hair treatment composition comprising a zinc-based antidandruff agent, a conazole fungicide and a cationic modified guar deposition polymer.

The present invention relates to a hair treatment composition comprising anti-dandruff agents and a cationic deposition polymer.

DE 101 11 288 (Wella) discloses a composition comprising climbazole, zinc pyrithione and Polyquaternium-10.

Despite the prior art there remains the need for improved anti-dandruff compositions.

Accordingly, the present invention provides a hair treatment composition comprising a zinc-based antidandruff agent, a conazole fungicide and a cationic modified guar deposition polymer.

Preferably the zinc-based antidandruff agent is zinc pyrithione.

Preferably, the zinc-based antidandruff agent is present at from 0.01 to 2% wt. of the composition. More preferably it is present at from 0.5 to 1.5 and most preferably from 0.75 to 1.25% wt. of the composition.

Preferably, the conazole fungicide is selected from ketoconazole and climbazole.

Preferably, the conazole fungicide is present at from 0.01 to 2% wt of the composition. More preferably it is present at from 0.025 to 0.75% wt. of the composition.

Such cationic deposition polymers may be a homopolymer or be formed from two or more types of monomers. The molecular weight of the polymer will generally be between 5000 and 10000000 unified atomic mass units, typically at least 10000 and preferably from 100000 to 2000. The polymers will have cationic nitrogen containing groups such as quaternary ammonium or protonated amino groups, or a mixture thereof. A suitable cationic deposition polymer is AQU D4051 a cationically-modified guar commercially available from Aqualon.

Preferably, the cationic deposition polymer is guar hydroxypropyl trimonium chloride.

It is highly preferred that compositions according to the invention should contain from 0.04% to 2% wt. of the composition cationic deposition polymer, more preferably from 0.05 to 0.5% wt. and most preferably from 0.08 to 0.25% by weight of the composition.

The composition according o the invention may be in any common product form used to treat hair. However, preferably, it is shampoo composition.

The composition according to the invention may comprise any of the ingredients commonly found in hair treatment compositions depending on the product form.

For example, where the composition is a shampoo it will comprise at least one cleansing surfactant such as is commonly employed in shampoos. Where it is a composition which aims to provide conditioning benefit, whether as part of a shampoo composition or a dedicated conditioning composition it will comprise a conditioning active. Suitable conditioning actives include fatty alcohols, silicones and cationic surfactants.

### EXAMPLE 1

The following formulation is an embodiment he invention and is made by standard processes.

| Ingredient | % wt. |
|---|---|
| Sodium Laureth Sulphate | 14 |
| Cocoamidopropyl betaine | 1.6 |
| Guar hydroxypropyl trimonium | 0.2 |
| chloride* | |
| Zinc Pyrithione | 1.0 |
| Climbazole | 0.5 |
| Zinc sulphate | 0.1 |
| Dimethicone | 2.0 |
| Perfume | 0.7 |
| Sodium chloride | 0.5 |
| Propylene Glocol | 1.0 |
| Acrylic Acid polymer (Carbomer) | 0.6 |
| Polypropylene Glycol | 0.1 |
| Preservative | 0.22 |
| Preservative | 0.09 |
| Water | To 100 |

*Jaguar C17

### EXAMPLE 2

The following demonstrates the efficacy of climbazole in the presence of cationic guar and polyquatemium-10.

| | Conc. ZPTO μg/cm3 after | |
|---|---|---|
| Test sample | one rinse (two rinses) | S.D. |
| 1% ZPTO, | 15.51 (12.199) | 1.35 (1.456) |
| 0.5% climbazole, | | |
| 0.2% Jaguar C17 | | |
| 1% ZPTO, | 12.39 (7.49) | 1.11 (0.57) |
| 0.5% climbazole, | | |
| 0.2% PQ-10 | | |

1. (canceled)

2. (canceled)

3. (canceled)

4. (canceled)

5. A method of enhancing deposition of zinc pyrithione in a shampoo composition comprising said zinc pyrithione, which method comprises combining the zinc pyrithione with climbazole and 0.05 to 0.5% by wt. guar hydroxypropyl trimonium chloride.
